(19) United States
(12) Patent Application Publication
(10) Pub. No.: US 2025/0176836 A1
(43) Pub. Date: Jun. 5, 2025

(54) SYSTEMS AND METHODS FOR REGISTRATION AND ACTIVATION OF TEMPERATURE-SENSING GARMENTS

(71) Applicant: Siren Care, Inc., San Francisco, CA (US)

(72) Inventors: Jie FU, Shanghai (CN); Ran MA, San Francisco, CA (US); Henk Jan Scholten, San Francisco, CA (US); Xuening SHEN, Shanghai (CN)

(21) Appl. No.: 18/825,630

(22) Filed: Sep. 5, 2024

Related U.S. Application Data
(63) Continuation of application No. 18/418,592, filed on Jan. 22, 2024, now abandoned.
Application No. 18/418,592 is a continuation of application No. 18/313,238, filed on May 5, 2023, now abandoned.
Application No. 18/313,238 is a continuation of application No. 16/381,818, filed on Apr. 11, 2019, now abandoned.
(60) Provisional application No. 62/656,336, filed on Apr. 11, 2018.

(51) Int. Cl. A61B 5/01; A43B 3/34; A61B 5/00; A61B 5/11

(52) U.S. Cl. CPC A61B 5/01; A43B 3/34; A61B 5/0008; A61B 5/0022; A61B 5/1126; A61B 5/6807; A61B 2562/0219; A61B 2562/0271

(57) ABSTRACT

A system for monitoring a patient includes a garment including a controller and one or more temperature sensors, and a receiving station configured to communicate with the garment over a wireless communication network. The garment may be operable in an inactive mode in which the controller samples the temperature sensors to generate a first set of temperature measurements, and an active mode in which the controller samples the temperature sensors to generate a second set of temperature measurements and the garment communicates the second set of temperature measurements to the receiving station, where the controller transitions the garment from the inactive mode to the active mode based at least in part on the first set of temperature measurements and/or other sensor data such as garment motion data and/or garment orientation data.

### CROSS-REFERENCE TO RELATED APPLICATIONS

This application is a continuation of U.S. patent application Ser. No. 18/418,592, filed Jan. 22, 2024, which is a continuation of U.S. patent application Ser. No. 18/313,238, filed May 5, 2023, which is a continuation of U.S. patent application Ser. No. 16/381,818, filed Apr. 11, 2019, which claims priority to U.S. Provisional Patent Application No. 62/656,336, filed Apr. 11, 2018, each of which is hereby incorporated in its entirety by this reference.

### TECHNICAL FIELD

This invention relates generally to the field of health care, and more specifically to new and useful temperature-sensing garments and systems and methods for registration and activation of the same.

### BACKGROUND

Certain diseases may result in nerve damage, leading to decreased sensation in the extremities of a patient. For example, diabetes (an increasingly common medical condition in which the body has an impaired ability to produce or respond to the hormone insulin) damages blood vessels and nerves, particularly in the feet, and can lead to severe medical complications that are difficult to treat. For example, one complication of poorly controlled diabetes is foot ulcers, which may fail to heal because of poor blood circulation in diabetics and because treatment does not always successfully halt the spread of infection. Diabetic foot ulcers are painful and, when unresolved, can lead to lower limb amputations. As another example, Charcot foot, also known as Charcot arthropathy, is a debilitating complication of diabetes involving fractures and dislocations of bones and joints that occur with minimal or no known trauma. Diabetics may also suffer from diabetic neuropathy (numbness or less of feeling as a result of nerve damage), which results in decreased sensation in the feet.

Self-care is critical to detecting early signs of ulcers, Charcot foot, and other injuries and allowing timely treatment. However, decreased sensation in the feet due to diabetic neuropathy may impair the ability to self-detect these injuries. Further, visual inspection for detecting such conditions has limitations. For example, obese or visually impaired patients may not be able to see their feet easily. Even further, X-rays are unable to reliably show early stages of fractures. Accordingly, painful and dangerous foot conditions may be detected only when they have progressed to a more severe state, which increases the likelihood of extreme treatments such as amputation. Current methods of detecting early signs of ulcers, Charcot foot, and other injuries are limited to self-inspection and periodic check-ups by a physician. As described above, self-inspection may be severely impaired for diabetic patients, and doctor visits are time consuming, inconvenient, and may not be a reliable method of early detection.

Thus, there is a need for new and improved systems and methods for monitoring the feet health of patients.

### SUMMARY

Generally, in some variations, a system for monitoring a patient includes a garment including a controller and one or more temperature sensors, and a receiving station configured to communicate with the garment over a wireless communication network. The garment may be operable in an inactive mode and an active mode. In the inactive mode, the controller may sample the one or more temperature sensors to generate a first set of temperature measurements. In the active mode, the controller may sample the one or more temperature sensors to generate a second set of temperature measurements, and the garment may communicate the second set of temperature measurements to the receiving station. In some variations, the controller transitions the garment from the inactive mode to the active mode based at least in part on the first set of temperature measurements.

In some variations, the controller may automatically determine placement of the garment on the patient and transition the garment from the inactive mode to the active mode in response to determining placement of the garment on the patient. For example, the controller may automatically determine placement of the garment on the patient based at least in part on a change within the first set of temperature measurements satisfying a predetermined temperature condition. For example, in some variations, the garment may include an orientation sensor, and the controller may automatically determine placement of the garment on the patient based at least in part on a detected orientation of the garment satisfying a predetermined orientation condition. Additionally or alternatively, in some variations, the garment may include a motion sensor, and the controller may automatically determine placement of the garment on the patient based at least in part on a detected motion of the garment satisfying a predetermined motion condition.

In some variations, when the garment is operated in the inactive mode, the controller may sample the one or more temperature sensors at a first nonzero frequency, and when the garment is operated in the active mode, the controller samples the one or more temperature sensors at a second frequency greater than the first frequency.

The garment may be configured to be worn on a foot of the patient. For example, in some variations, the garment may be a sock. In some variations, the system may include a first garment configured to be worn on a first foot (e.g., left foot) of the patient, and a second garment configured to be worn on a second foot (e.g., second foot) of the patient. At least one of the first garment and the second garment may include a plurality of temperature sensors arranged on a sole region of the garment.

In some variations, the receiving station may further include a wireless communication module configured to communicate temperature measurements to a remote computing system (e.g., cloud-based server) over a wireless communication network. The receiving station may include a housing and a power supply, and the housing may include a visual status indicator (e.g., one or more colored LEDs).

Generally, in some variations, a method for monitoring a patient may include pairing a garment (e.g., sock) to at least one electronic device, wherein the garment comprises one or more temperature sensors, and generating a first set of temperature measurements in an inactive mode using the one or more temperature sensors. The method may additionally include, in response to a determination that the garment is placed on the patient, generating a second set of temperature measurements in an active mode using the one or more temperature sensors, and wirelessly communicating the second set of temperature measurements to the at least one electronic device. In some variations, generating a first set of temperature measurements in the inactive mode may include sampling the one or more temperature sensors at a first nonzero frequency, and generating a second set of temperature measurements in the active mode may include sampling the one or more temperature sensors at a second frequency greater than the first frequency.

The method may further include wirelessly communicating the second set of temperature measurements to the at least one electronic device. Examples of the paired electronic device include a receiving station (e.g., hub) and a mobile computing device (which may, for example, be executing a native health application). In some variations, the method may further include wirelessly communicating the second set of temperature measurements from the electronic device to a remote computing system (e.g., cloud-based server).

In some variations, pairing the garment to the electronic device may include pairing the garment to the electronic device using a garment identifier associated with the garment. For example, pairing may include receiving a serial code or an image of a barcode label (e.g., QR code). Furthermore, in some variations, the method may include pairing a second garment to the at least one electronic device, wherein the second garment may include one or more temperature sensors. For example, at least two garments (e.g., a left foot garment and a right foot garment) may be simultaneously paired to an electronic device (or multiple electronic devices).

In some variations, the method may further include determining that the garment is placed on the patient. This determination may be made in a combination of one or more various manners. For example, in some variations, the determination that the garment is placed on the patient may be based at least in part on detected motion of the garment, detected orientation of the garment, or both. As another example, the determination that garment is placed on the patient may be based at least in part on the first set of temperature measurements obtained in the garment inactive mode (e.g., by determining that a temperature change satisfies a predetermined temperature condition). Exemplary temperature conditions include rising temperature measurements, at least a threshold rate of change in temperature measurements, and an absolute value of temperature measurements performed by one or more temperature measurements in the garment.

Generally, in some variations, a method for automatically determining, without user input, whether a garment is placed on a user may include detecting an orientation of the garment using one or more orientation sensors in the garment, receiving a set of garment temperature measurements from one or more temperature sensors in the garment, and determining that the garment is placed on a user based at least in part on the detected orientation of the garment and the received garment temperature measurements. For example, determining that the garment is placed on a user may be based at least in part on the detected orientation of the garment satisfying a predetermined orientation condition. Additionally or alternatively, determining that the garment is placed on a user may include determining a change in the garment temperature measurements (e.g., over a sampling period) and determining that the garment is placed on a user may be based at least in part on the determined change in the garment temperature measurements (e.g., a determined rise in temperature measurements over a sampling period, a rate of change, etc.). Furthermore, in some variations the method may include detecting motion of the garment using one or more motion sensors in the garment, and determining that the garment is placed on a user may be based at least in part on the detected motion of the garment.

### BRIEF DESCRIPTION OF THE DRAWINGS

FIG. 1 is an illustrative schematic depicting a system for monitoring health of one or more patients.

FIGS. 2A and 2B are illustrative schematics depicting an exemplary variation of a garment with sensors.

FIG. 3 is an illustrative schematic of a mobile computing device having a memory device including a health monitoring application.

FIG. 4 is an illustrative schematic of a receiving station in a system for monitoring health of a patient.

FIGS. 5A and 5B depict an exemplary variation of a receiving station in a system.

FIG. 6 is a flowchart depicting an exemplary variation of a method for monitoring health of a patient.

FIG. 7 is another flowchart depicting an exemplary variation of a method for monitoring health of a patient.

FIG. 8 is an illustrative schematic depicting an exemplary variation of a method including registering and activating a garment for monitoring health of a patient.

FIG. 9 is an illustrative schematic depicting an exemplary variation of a method including providing timestamps for sensor data generated by a garment for monitoring health of a patient.

FIG. 10 is an illustrative schematic depicting an exemplary of a method for monitoring health of a patient.

FIG. 11 is an illustrative schematic depicting an exemplary variation of display of sensor data and notifications in a method for monitoring health of a patient.

FIG. 12 is another illustrative schematic depicting an exemplary variation of a method for monitoring health of a patient.

### DETAILED DESCRIPTION

Non-limiting examples of various aspects and variations of the invention are described herein and illustrated in the accompanying drawings.

Described herein are systems and methods for registering and/or activating garments having one or more sensors, such as temperature-sensing garments. For example, such systems and methods may enable user-friendly, efficient collection and communication of sensor data from a garment configured to be worn by a patient, where sensor data may be subsequently analyzed for patient monitoring of a health condition.

### Systems for Patient Monitoring

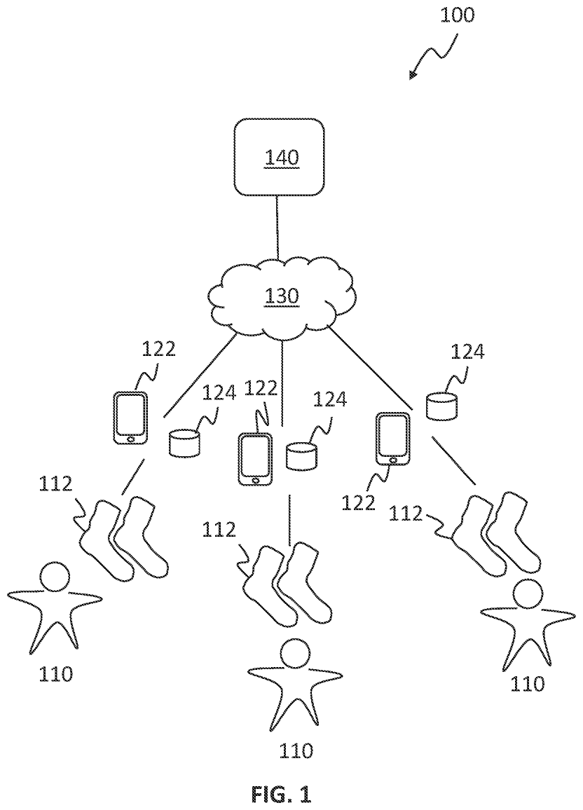

As shown in FIG. 1, a monitoring system 100 may be configured to monitor one or more patients 110 or other users. For example, for each patient, the monitoring system 100 may include one or more garments 112 that include one or more sensors generating sensor data, and one or more electronic devices for communicating sensor data from the garment sensor(s) over a wireless communication network (e.g., link) to a remote computing system 140. The sensor data may include information that may be analyzed to determine, for example, one or more biometrics (e.g., temperature) of the patient, a status of the garment (e.g., position, orientation, motion, etc.), and/or any suitable information about the garment or patient. The electronic device receiving sensor data may be, for example, a mobile computing device 122 (e.g., mobile phone, tablet, etc.) or a receiving station 124 such as that described in further detail below. Furthermore, the one or more electronic devices may be configured to communicate sensor data to at least one remote computing system 140 (e.g., server) over a wireless communication network 130. Alternatively, in some variations, sensor data may be communicated from the one or more garments 112 directly to a remote computing system 140. Some or all of the sensor data from the garments 112 may be analyzed by one or more processors, such as in the mobile computing device 122, receiving station 124, and/or remote computing system 140, as further described below.

In some variations, the monitoring system 100 can assist in diagnosing and/or predicting health conditions in a patient. For example, patients with diabetic neuropathy (which may cause loss of feeling in patients' feet) may be unable to identify a sore or infection formed on their feet and/or periods of poor blood circulation through their feet, which may lead to debilitating infections and, ultimately, to amputation. When a region of a foot becomes infected, the temperature of this region of the foot may rise as the body combats this infection. While a diabetic patient may be less able to feel pain from this infection, temperature-sensing garments (e.g., garments, slippers, shoes, shoe inserts, or other soft goods worn by the patient) may read temperatures of various regions across the patient's feet. The temperature measurements may be used to predict and/or detect this infection, such as before the infection becomes so severe that the patient risks losing the infected foot. Exemplary methods for assessing foot inflammation based on foot temperature measurements from the system are described in U.S. Patent App. Pub. No. 2017/0188841, which is hereby incorporated in its entirety by this reference.

### Garment

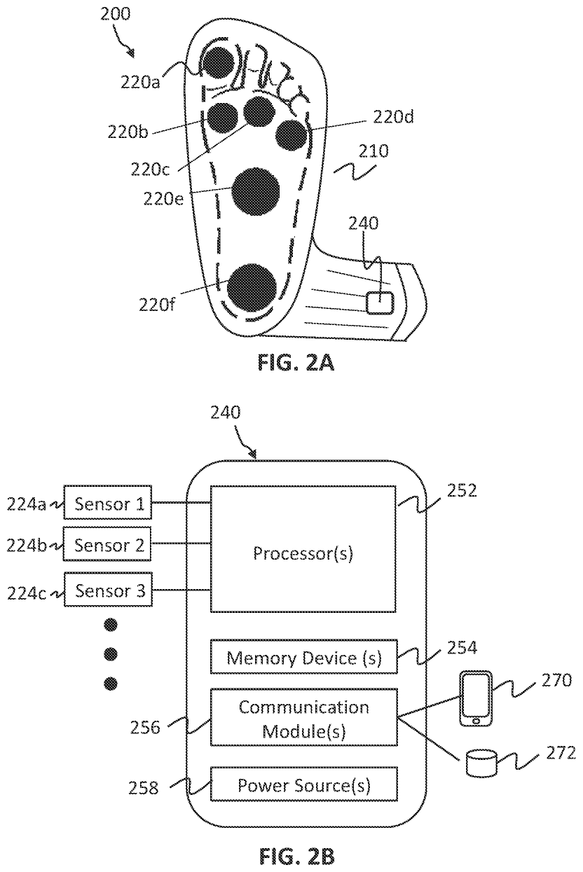

As shown generally in FIG. 2A, in some variations, a monitoring system may include at least one garment 200 configured to be placed or worn on a foot of the patient. For example, the garment may be a sock, a shoe, a slipper, an insole, etc. In some variations, the garment may be configured specifically for a left foot (e.g., include a toe box accommodating contours of a left foot), for a right foot (e.g., include a toe box accommodating contours of a right foot), or may be universal or suitable for either foot. The garment may include one or more labels that are sewn, woven, or otherwise incorporated into or coupled to the garment. Examples of labels include an indication of left foot or right foot compatibility, size (e.g., small, medium, large, or numeric size), or other identifying information. Although the garment is primarily described herein as a garment configured to be placed or worn on a foot of a patient, it should be understood that in other variations, a garment may be configured to be placed or worn on any other suitable part of a patient (e.g., leg, arm, hand, neck, torso, head, etc.).

The garment 200 may be configured to be worn on a foot (e.g., of a human) and include a set of one or more sensors integrated into or otherwise embedded or attached to the garment. The sensors may be configured to measure one or more physical characteristics of the patient, such as skin temperatures at one or more locations on a foot of the patient. In some variations, a patient may wear two garments, including one garment on a left foot of the patient, and another garment on a right foot of the patient. Sensor measurements may be performed substantially continuously as the garments are worn, thereby providing continuous monitoring of health (e.g., foot health) of the patient. For example, one or more sensors in the garment 200 may be read or scanned periodically, such as every second, every 10 seconds, every 30 seconds, every minute, every hour, or other suitable interval). In some variations, the garment can be paired (i.e., communicatively coupled) with a matching garment (e.g., “a right garment”) including similar components to form a pair of garments (e.g., a pair of left and right garments).

The one or more sensors may be integrated or otherwise embedded or attached to the garment in any suitable manner. For example, one or more sensors may be arranged in a pocket or recess constructed in the garment, or affixed to a surface (e.g., internal or external surface) of the garment such as by adhesive, sewing, or the like. Exemplary methods of making sensing garments such as temperature-sensing garments, for example, are described in International Patent App. Nos. PCT/CN2018/121244 and PCT/US2019/018714, each of which is hereby incorporated in its entirety by this reference.

In the example shown in FIG. 2A, the garment 200 may include one or more temperature sensors (e.g., thermistor, thermocouple) arranged in a configuration or pattern and enabling collection of temperature values at multiple target regions of a foot placed within the garment 200. For example, a left garment can include six temperature sensors arranged in a left pattern with: a first temperature sensor 220a positioned in a first region of the garment configured to face the first Ossa digit proximal the distal phalange of a patient's left foot when the left garment is worn by the patient; second, third, and fourth temperature sensors (220b, 220c, 220d) positioned across a second region of the garment configured to face the boundary of the phalanges and the metatarsals of the patient's left foot when the left garment is worn by the patient; a fifth temperature sensor 220e positioned in a third region of the garment configured to face the boundary of the metatarsals and the tarsals of the patient's left foot when the left garment is worn; and a sixth temperature sensor 220f positioned in a fourth region of the garment configured to face the heel of the patient's left foot when the left garment is worn by the patient. Accordingly, the left garment can include a set of temperature sensors distributed across four distinct temperature zones of the patient's foot. A right garment can similarly include a set of six temperature sensors arranged in a right pattern mirroring the left pattern. However, the sensors may be arranged in other suitable patterns (e.g., depending on type of garment, type of health condition being monitored, etc.).

In other variations, one or more sensors (e.g., temperature sensors, pressure sensors, pulse-oximeter sensors, etc.) may additionally or alternatively be arranged on other regions of the garment (e.g., medial or lateral sides of a foot region of the garment, a dorsal portion of a foot region of the garment, an ankle region of the garment, etc.). For example, one or more temperature sensors may be arranged on the garment so as to measure skin temperature of the ankle and/or leg (e.g., lower leg).

In some variations, the garment may further include at least one housing 240. As shown in FIG. 2B, the housing 240 may house an electronic subsystem for receiving data (e.g., from sensor leads) from the sensors, and/or house other various components. The housing 240 may, for example, be arranged on an ankle region of the garment. In some variations, the housing may be secured to the garment by being enclosed between the garment and a cover. As another example, the garment can include a pocket configured to receive the housing, and channels configured to receive wires extending from the housing to each sensor in the set of sensors. For example, the pocket or pouch can be sewn into a neck or ankle portion (e.g., upper portion) of a foot garment (e.g., sock) during manufacturing. In another example, a foot garment can include channels sewn along a side of a patient's foot when the garment is worn by the patient, such that the patient may not feel the wires within the garment when the patient wears the garment. Additionally or alternatively, the housing may be coupled to the garment with an adhesive or mechanical fasteners (e.g., rivets, snaps, etc.), sewn to the garment, or in any suitable manner.

The housing may be substantially sealed (e.g., hermetically sealed or waterproofed) to protect the contents of the housing from environmental conditions (e.g., when the garment is washed or worn, when the patient is sweating, etc.). For example, at least a portion of the housing may be filled with and/or otherwise sealed with a substance adhering to the housing, such as ultraviolet glue, silicone, other epoxy or polymer, etc. As another example, the housing may include one or more components that may be coupled together (e.g., with a suitable mechanical interfit, fasteners, weld, etc.). The joint between coupled housing components may be sealed by epoxy or other suitable sealant. In some variations, the one or more components of the housing may be formed at least in part through injection molding.

As shown in the schematic of FIG. 2B, the housing 240 may house various components for operating the system. For example, the housing 240 may include at least one processor 252 (e.g., CPU), at least one memory device 254 (which can include one or more computer-readable storage mediums), at least one communication module 256, and at least one power source 258. In some variations, the housing may further include one or more additional activity or other sensors (e.g., an accelerometer, a gyroscope, or an inertial measurement unit). One or more of these components may be arranged on one or more electronic circuit boards (e.g., PCBA), which in turn may be mounted to the housing.

The processor 252 and memory device 254 may cooperate to provide a controller for operating the system. For example, the processor 252 may receive sensor data from one or more sensors (e.g., first sensor 224a, second sensor 224b, third sensor 224c, etc.), and the sensor data may be stored locally in the one or more memory devices 254. In some variations, the processor 252 and memory 254 may be implemented on a single chip, while in other variations they can be implemented on separate chips. In some variations, the controller may be similar to the controller described in U.S. Patent Pub. No. 2017/0188841, which was incorporated by reference above. Other exemplary aspects of the controller are described herein.

Generally, the controller can operate the garment in an inactive mode and in an active mode. The controller may operate the garment in the inactive mode when there is an indication that the garment is not being worn by the patient, and may operate the garment in the active mode when there is an indication that the garment is placed on (e.g., worn) by the patient. For example, in the inactive mode, the controller may scan at least some of the one or more garment sensors at a relatively low frequency (e.g., a nonzero frequency that may be sufficient to receive and store sensor data for use in determining whether the garment is being worn, for example). When the garment is in the active mode, the controller may, for example, operate the garment in a manner that produces sensor data sufficient for actively monitoring a health condition. For example, the controller may enable sensor data to be received, processed, stored in the memory device 254, and/or broadcast to an external electronic device (e.g., mobile computing device, receiving station, etc.) via the communication module 256. In the active mode, the controller may scan at least some of the one or more garment sensors to receive and store sensor data at a relatively high frequency.

In some variations, sensor measurements obtained when the garment is in the inactive mode may be distinguishable from that obtained when the garment is in the active mode (e.g., tagged differently such as with a meaningful timestamp, or discarded, etc.), such that analysis of the patient condition may rely upon only data obtained when the garment is in the active mode and placed on the patient. Accordingly, the analysis may be more accurate and lead to more effective patient monitoring. Furthermore, when the garment is in the inactive mode, the controller may, for example, operate the garment in a manner that reduces depletion of the power source 285 (e.g., due to reduced frequency of sensor scanning, which takes less energy) when the garment is being stored, washed, or otherwise not worn.

In some variations, the controller may toggle or transition between the inactive mode and the active mode based on sensor data suggesting whether the garment is placed on a patient (e.g., processing of data from activity sensors, temperature sensors, etc.). For example, the controller may be configured to determine, without patient input (e.g., pressing of a button or other confirmation by the patient) whether the garment is placed on a patient. For example, as further described below, a garment can remain in an inactive mode as a default (e.g., based on an assumption that majority of the time, the garment is not placed on a patient) but may transition to the active mode based on one or more sensor signal inputs suggesting that the garment is placed by a patient. Variations of methods of activation and registration of the garment are described in further detail below.

The communication module 256 may be configured to communicate sensor data and/or other information to an external computing device. In some variations, the external computing device may be a mobile computing device 270 (e.g., mobile telephone, tablet, smart watch, laptop etc.). Additionally or alternatively, the external computing device 270 may be, for example, a receiving station 272 (e.g., hub) such as that described herein, a desktop, or other suitable computing device. The external computing device 270 may be one or more networked devices, such as a mobile computing device or receiving station paired with the garment, a server, a cloud network, etc.

The communication module 256 may communicate via a wireless network (e.g., through NFC, BLUETOOTH, WIFI, RFID, or any type of digital network that is not connected by cables). Alternatively, in some variations, the communication module 256 may communicate via a wired connection (e.g., including a physical connection such as a cable with a suitable connection interface such as USB, mini-USB, etc.), such as to an electronic device (e.g., mobile computing device, receiving station, etc.). For example, devices may directly communicate with each other in pairwise connection (1:1 relationship), or in a hub-spoke or broadcasting connection (“one to many” or 1:m relationship). As another example, the devices may communicate with each other through mesh networking connections (e.g., “many to many”, or m:m relationships), such as through BLUETOOTH mesh networking. Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), BLUETOOTH, Wireless Fidelity (WIFI) (e.g., IEEE 802.11a, IEEE 802.11b, IEEE 802.11g, IEEE 802.11n, and the like), or any other suitable communication protocol. Some wireless network deployments may combine networks from multiple cellular networks or use a mix of cellular, WIFI, and satellite communication.

Additionally, the housing in the garment may include one or more power sources 258, which may function to provide electrical power to the processor, communication module, sensors, and/or any other electrical components. For example, the power source 258 may include one or more batteries. In some variations, the power source 258 may be rechargeable such as through wireless charging methods (e.g., inductive charging, RF coupling, etc.) or by harnessing kinetic energy such as that generated through motion (e.g., when the patient walks while wearing the garment).

### Mobile Computing Device

As described above, the communication module 256 in a garment may be configured to communicate sensor data and/or other information to a mobile computing device, which may be, for example, a mobile telephone, tablet, smart watch, laptop, or other suitable mobile computing device. The mobile computing device may receive sensor data and/or other information, and subsequently upload or otherwise transmit this data and/or other information to one or more remote computing systems through a suitable network connection (e.g. WIFI) for analysis and/or synchronizing to the patient's profile (which may include health status, medical history, trends in sensor data, etc.).

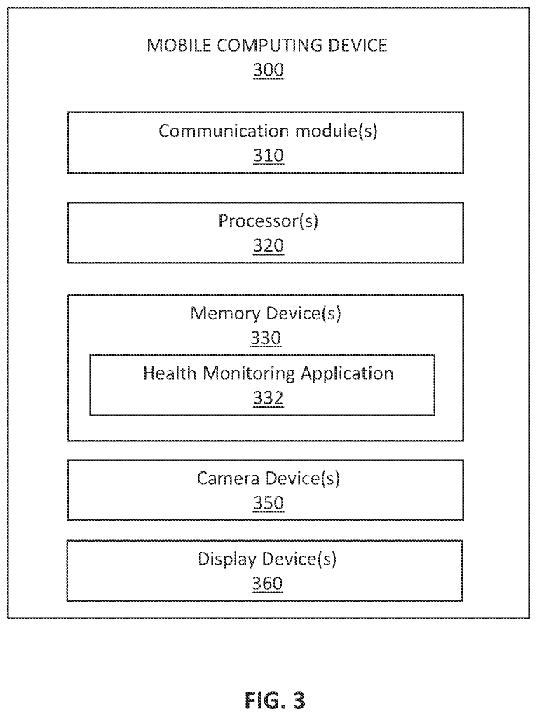

FIG. 3 illustrates an exemplary schematic of a mobile computing device 300 for communicating with the garment. Generally, in some variations, the mobile computing device 300 may include a communication module 310 (which may be similar in construction to the communication module 256 in the garment as described above), and a controller including at least one processor 320 (e.g., CPU) and at least one memory device 330 (which can include one or more computer-readable storage mediums).

The processor 320 may incorporate data received from the memory device 330. For example, the memory device 330 may store a native health monitoring application 332 for execution by the controller. In some variations, the health monitoring application 332 may facilitate and/or regulate a communication link between one or more sensing garments and the mobile computing device. In an exemplary variation, sensor data from the one or more sensing garments may be broadcast to the mobile computing device through a wireless communication link (e.g., BLUETOOTH), and subsequently transmitted to one or more remote computing systems through a wireless network connection (e.g., WIFI).

Additionally or alternatively, the health monitoring application 332 may present sensor data (and/or the results of analysis thereof) through a user interface. For example, data and/or results of analysis (e.g., notifications, alerts, etc.) may be displayed on a display device 360. In some variations, the display device 360 may include, for example, at least one of a light emitting diode (LED), liquid crystal display (LCD), electroluminescent display (ELD), plasma display panel (PDP), thin film transistor (TFT), organic light emitting diodes (OLED), electronica paper/e-ink display, laser display, holographic display, or any suitable kind of display device. In some variations, the health monitoring application 332 may further provide information via one or more input/output devices, such as one or more camera devices 350, one or more audio devices, haptic devices, etc. A camera device 350 may, for example, enable the patient to generate images (e.g., still images, videos, etc.) such as of body parts, for medical record-keeping (e.g., historical reference, remote visual diagnosis, etc.). In some variations, an audio device may include at least one of a speaker, a piezoelectric audio device, magnetostrictive speaker, and/or digital speaker. Other output devices may include, for example, a vibration motor to provide vibrational feedback to the patient (e.g., for an alert or notification). Other aspects of the native health monitoring application 332 are described in further detail below.

### Receiving Station

As described above, the communication module 256 in a garment may be configured to communicate to a receiving station (e.g., hub). In some variations, the receiving station may be paired (e.g., communicatively coupled) with one or more sensing garments to receive sensor data generated by the sensors in the garments (e.g., temperature data). The receiving station may further be configured to communicate sensor data through a network to one or more remote computing systems (e.g., server). The receiving station may be, for example, a networked device that may be standalone and configured to receive and/or send sensor data and/or other information (e.g., timestamps accompanying sensor data) through a network (e.g., cloud network) as described above.

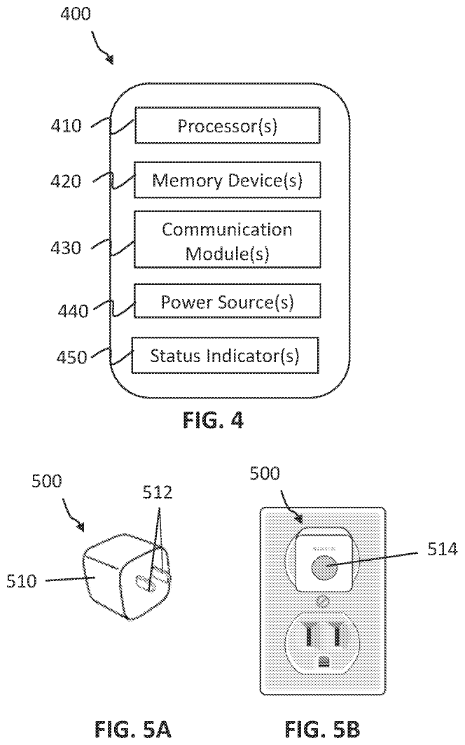

Generally, as shown in FIG. 4, a receiving station 400 may include one or more processors 410, one or more memory devices 420, and one or more communication modules 430, which may physically be similar to those described above for the garment and/or mobile computing device. For example, the processor 410 and memory device 420 may cooperate to provide a controller for operating the receiving station 400. Sensor data and/or other information received from the garment(s) may be stored in the one or more memory devices 420, and upload and synchronized via a network to the patient's profile. In an exemplary variation, sensor data is received from the one or more garments through a wireless communication link (e.g., BLUETOOTH), and subsequently transmitted to one or more remote computing systems through a wireless network connection (e.g., WIFI). In variations in which the system includes both a mobile computing device and a receiving station, one of the mobile computing device and the receiving station may be a primary receiver of sensor data, while the other may be a secondary receiver of sensor data in that the secondary receiver only receives data if the primary receiver is not connected or otherwise incapacitated. For example, in some variations, a health monitoring application 332 may not be “open” on the mobile computing device, thereby hampering connectivity between the garment and the mobile computing device. In this scenario, the receiving station may instead receive sensor data from the garment (e.g., if in suitable proximity, as described below) and/or the sensor data may be stored locally on the garment until connection with the mobile computing device is established or re-established. Accordingly, in some variations, the combination of a mobile computing device and a standalone receiving station (each individually capable of receiving and transmitting sensor data) may help keep the record of uploaded sensor data at the one or more remote computing systems as up-to-date and accurate as possible. Thus, the system may provide the patient with accurate and timely notifications and alerts associated with analysis of the sensor data.

In some variations, as shown in FIG. 4, the receiving station 400 may include one or more status indicators 450 configured to communicate an operational status of the receiving station to the patient or other users. For example, the status indicator 450 may provide information regarding connectivity of the receiving station over a communication link to one or more garments, and/or to a network to one or more remote devices. As another example, the status indicator 450 may provide information regarding power supplied to the receiving station 400, and/or other operational conditions such as temperature of the receiving station 400, so as to warn a user about lack of power, overheating of the receiving station, etc. In some variations, the status indicator 450 may be a visual status indicator. For example, the receiving station may include a housing with at least one light source (e.g., LEDs) forming at least part of the status indicator 450. In this example, the light source may illuminate in different manners, such as by varying in color and/or temporal illumination patterns in order to communicate different statuses.

Additionally, the receiving station 400 may include one or more power sources 440 for providing electrical power to the various electrical components in the receiving station, including the processor, memory, communication module, status indicator, etc. A power source 440 may be internal to or integrated in the receiving station (e.g., battery), or may include at least one connection suitable for sourcing power from an external entity (e.g., wall outlet connected to a power grid, port for a power cable, etc.).

FIGS. 5A and 5B illustrate an exemplary variation of a receiving station 500, or hub. As shown in FIG. 5A, the receiving station 500 includes a housing 510 configured to plug into a wall power outlet via outlet prongs 512 (FIG. 5B). In some variations, outlet prongs 512 for sourcing power from an external power grid may provide a more reliable and consistent source of power, and accordingly may be advantageous by reducing the risk of missing sensor data as the result of loss of power to the receiving station 500.

The housing 510 as shown in FIGS. 5A and 5B is generally prismatic (e.g., somewhat cube-like); however, it should be understood that the housing 510 may take any suitable form. On an exterior surface of the housing 510, a visual status indicator 514 includes a plurality of LEDs of multiple colors. In some variations, each LED may be optically coupled to an optical waveguide that carries light to a particular feature (e.g., around a perimeter of the receiving station) via internal reflection. In these variations, the optical waveguide may appear to be different colors depending on the nature of the illuminated LED. Alternatively, the LEDs may be arranged in an array (e.g., a row) for selective illumination without the use of an optical waveguide. Selectivity and timing of illumination of LEDs may be operated by the controller in the receiving station, such that different selection of color(s) and/or blinking patterns may indicate different statuses of the receiving station.

For example, the receiving station 500 may include at least four different colors of LEDs (blue, red, yellow, and green) and at least two timing schemes (non-blinking and blinking), the combination of which allows for at least eight distinct illumination appearances. For example, a solid (non-blinking) blue appearance may indicate that the receiving station is not connected to a network for uploading sensor data (e.g., to cloud storage). As another example, a blinking red appearance may indicate another problem associated with the network. Other schemes may indicate connectivity status to zero garments (blinking blue appearance), one garment (blinking yellow appearance), two garments (blinking green appearance), etc. being successfully communicatively connected to the receiving station. It should be understood that these illumination schemes are only exemplary and other illumination schemes may be correlated to any suitable kind of status of the receiving station.

However, in other variations, the receiving station may be any suitable networked device configured to receive signals from the one or more garments and/or other electronics devices. For example, the receiving station may be a home virtual assistant device (e.g., similar to Amazon Echo® or Google Home™ devices), a set top box service (e.g., similar to Apple TV®), or other smart appliances such as a clock, radio, and the like.

### Methods for Monitoring

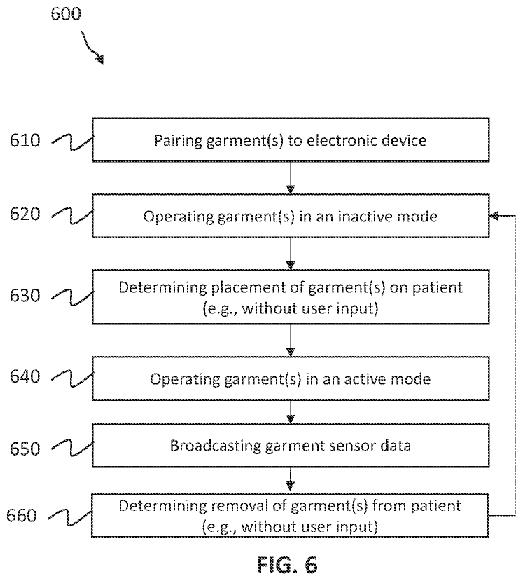

Generally, as shown in FIG. 6, a method 600 for monitoring a patient involves pairing at least one sensing garment to an electronic device 610. The sensing garment may be similar to the garment described above, and electronic device 610 may be, for example, a mobile computing device (e.g., mobile telephone) and/or receiving station as also described above. The method 600 may further include operating the garment in an inactive mode 620, determining placement of the garment on the patient 630, operating the garment in an active mode 640 in response to determining placement of the garment on the patient, and broadcasting garment sensor data 650. In some variations, the method 600 may further include determining removal of the garment from the patient 660, and returning to operating the garment in the inactive mode 620 in response to determining removal of the garment from the patient. By transitioning back and forth between the inactive mode when the sensing garment is determined to not be worn, and the active mode when the sensing garment is determined to be worn, the method 600 may help distinguish between sensor data not representative of the patient condition (because the garment is not being worn when the data is gathered) and sensor data that is useful for analysis of the patient condition (because the garment is being worn when the data is gathered). Additionally, accurate identification and operational adjustments may conserve power usage of a power supply in the garment, thereby extending operational life of the power supply (and garment).

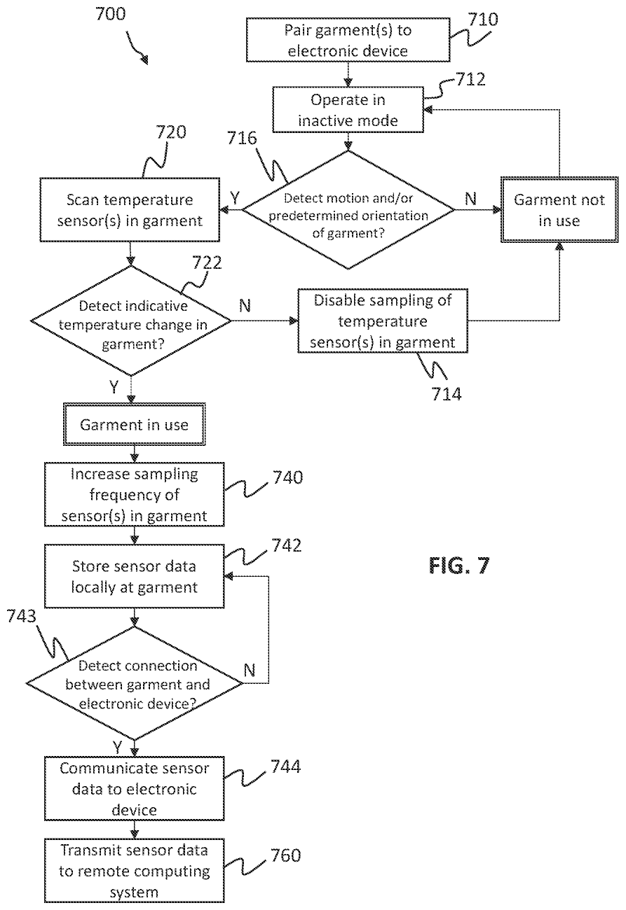

At least some portions of the methods described herein may be executed by a controller on the garment(s), an electronic device in communication with the garment(s) such as a mobile computing device (e.g., a smartphone, smartwatch, tablet, etc.) or a receiving station (e.g., hub, beacon, dongle, etc.). As described above, the electronic device may be configured to broadcast data over a wireless and/or cellular network, or any other device communicatively coupled to the garment via wireless communication protocols. Additional aspects of the methods are described in further detail below with reference to FIGS. 7-11. Although certain aspects of the methods for monitoring are described with respect to temperature-sensing garments, it should be understood that at least some portions of the methods may be applied to garments with other kinds of sensors.

### Pairing

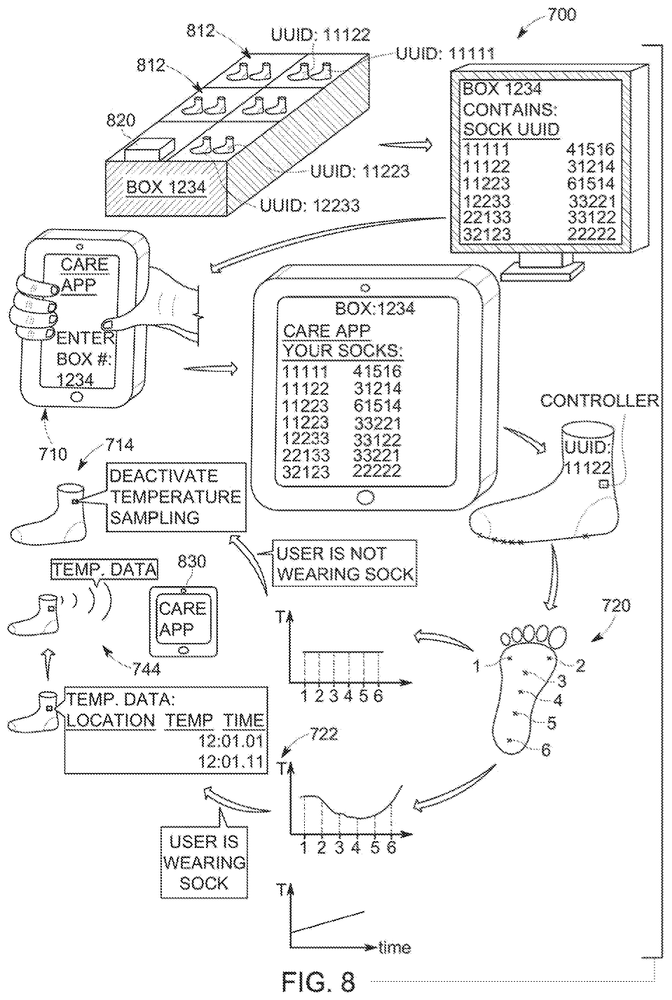

As shown in FIGS. 7 and 8, a method 700 for monitoring a patient includes pairing one or more garments to at least one electronic device (710). The electronic device(s) may include a receiving station and/or a mobile computing device, and/or other suitable electronic device. Exemplary aspects of pairing are described in further detail below.

### Pairing: Garment to Receiving Station

In some variations, pairing one or more garments to at least one electronic device (710) may include pairing each garment (e.g., in a set of temperature-sensing garments) to a receiving station. Generally, the controller of the garment may link or pair to a receiving station such that the controller may intermittently transmit sensor data (e.g., temperature measurements) to the receiving station, which may transmit relevant sensor data to one or more remote computing systems, such as for analysis in monitoring for a health or medical condition.

In some variations, each garment may be paired to a receiving station using a garment identifier associated with a garment, such as a unique user ID (e.g., a UUID), barcode, etc. For example, as illustrated in FIG. 8, a kit for monitoring a patient may include a set of one or more garments 812 and a receiving station 820. For example, a kit may include a set of five pairs of temperature-sensing garments and a receiving station. Prior to or during preparation of the kit (e.g., prior to shipment of the set of garments to a user), an operator may scan and record the garment identifier for each garment in the set of garments. The garment identifiers of each garment may be uploaded to the receiving station, such that the receiving station is configured to receive signals from each garment, thereby pairing each garment in the set of garments to the receiving station. Accordingly, the receiving station may receive data from and/or transmit data to each paired garment, at least under certain conditions (e.g., if the garment is within sufficient proximity to the receiving station, as described below).

Alternatively, prior to or during preparation of the kit, one or more garments in a kit may be paired to a receiving station by associating a kit identifier with the receiving station (e.g., similar to that described below with respect to pairing one or more garments to a mobile computing device).

In some variations, one or more garments may additionally (e.g., as confirmation) or alternatively be paired to a receiving station after the kit is shipped to a patient (e.g., as part of a system setup or after a determination that the garment is placed on a patient, etc.), based on individual garment identifiers and/or kit identifier associated with the kit.

Furthermore, in some variations, a garment may be paired with multiple receiving stations. For example, a patient may be provided with multiple receiving stations which may be placed in multiple locations, such as those frequented by the patient (e.g., living room, family room, home office, dining room, kitchen, etc. of the patient's residence, and/or the patient's workplace, etc.). For example, a garment paired with multiple receiving stations may be configured to communicate sensor data to the nearest receiving station within range.

Additionally or alternatively, the receiving station may be linked to or otherwise affiliated with a patient profile hosted by a remote computer system and accessible by the patient, other user, and/or a care provider (e.g., via a user web portal, native application, web browser, etc.). For example, after pairing a garment to a particular receiving station linked to a particular patient, the receiving station may directly import or upload sensor data recorded by paired garment into the patient profile. Such sensor data and/or other information may then be accessible by accessing the patient profile.

### Pairing: Garment(s) to Mobile Computing Device

In some variations, pairing one or more garments to at least one electronic device (710) may include pairing each garment (e.g., in a set of temperature-sensing garments) to a mobile computing device, such as a smart phone, tablet, smart watch, etc. over a communication network (e.g., wireless communication network). Garments may be paired to a mobile computing device collectively as a set of garments, and/or individually on a garment-by-garment basis.

In some variations, pairing of a set of garments may utilize a kit identifier, which may enable simultaneous pairing of multiple garments in a kit (or alternatively a single garment in a kit, as another method of individual pairing). For example, one or more garment identifiers for individual garments in a kit may be associated with a kit identifier, and the kit identifier may be paired with a mobile computing device to collectively associate the set of garments in the kit with the mobile computing device. In an illustrative example, prior to shipment of a kit including a set of one or more garments (e.g., to a patient, other user, distributor, reseller, etc.), the garment identifier of each garment in the kit may be assigned or otherwise associated with a unique kit identifier (e.g., QR code, other barcode, serial number, etc.). The unique kit identifier may be packaged with the kit, such as printed on a box containing the kit, printed on a label (e.g., adhesive label) affixed to the box, and/or included on a placard or other insert packaged within or attached to the box. The association of each garment identifier to the kit identifier may be uploaded to a database (e.g., a remote naming server).

In connection with receiving and/or using the kit, a recipient of the kit may download a native mobile application (e.g., health monitoring application) onto a mobile computing device. The native application on the mobile computing device may, in some variations, prompt the user to pair the kit identifier to a patient profile, thereby pairing each garment to the mobile computing device. For example, the native application may prompt a patient to log into a patient profile rendered within the native application, and enter the kit identifier associated with the kit of garments. The kit identifier may be entered into the native application in any suitable manner. For example, a kit identifier that is in the form of a QR code or other bar code may be entered by scanning with a camera device on the mobile computing device. A kit identifier that is in the form of a serial code may be entered by manual input (e.g., on a keyboard displayed on the mobile computing device), by scanning through optical character recognition techniques using a camera device on the mobile computing device, by interpreting dictation with a microphone device on the mobile computing device, etc. After the kit identifier is entered into the native application, the kit identifier may be linked or otherwise associated with the patient profile, thereby pairing the kit of garments with the mobile computing device.

Additionally or alternatively, some variations, each garment may be individually paired (on a garment-to-garment basis) to a mobile computing device based on individual garment identifiers (e.g., UUIDs) in a similar fashion as that described above.

Furthermore, in some variations, one or more garments may additionally (e.g., as confirmation) or alternatively be paired to a computing device after it is determined that the garments are placed on a user. Furthermore, the garment may pair to the mobile computing device (and/or any other device) in any other suitable way.

Once paired with the mobile computing device, each garment may wirelessly broadcast sensor data directly to the native application executing on the mobile computing device (e.g., via BLUETOOTH or another suitable wireless communication protocol), when the garment is within communication distance or range of the mobile computing device. As further described below, the native application may then automatically or semi-automatically (e.g., based on user command or other input) upload the sensor data to the patient profile.

### Pairing: Mobile Computing Device to Receiving Station

Furthermore, a mobile computing device may be paired with a receiving station, such as via a cellular (e.g., 3G, 5G, LTE, etc.) network and/or a local wireless computer network. For example, in some variations, the mobile computing device may facilitate a communication link between the receiving station and a patient profile hosted by a remote computer system. For example, a native application executed on the mobile computing device may link or register the receiving station to a patient profile hosted by a remote computer system by accessing an address (e.g., mechanical and/or wireless address) of the receiving device and registering the address to the patient profile. Accordingly, by linking the mobile computing device and the receiving station, the native application may link the receiving station directly to the user profile, such that the receiving station may directly upload sensor data to the patient profile (e.g., in the absence of the mobile computing device).

### Pairing: Garment to Garment

In some variations, garments may wirelessly pair with each other. For example, a left garment may wirelessly pair with a right garment, such as in an instance in which a mobile computing device is not within wireless range of the left and right garments. In this example, the controller in one of the paired garments may function as a master controller, and the controller in the other of the paired garments may function as a slave controller. Accordingly, the master controller may transmit one or more commands to the slave controller, such as for sensor scanning synchronization purposes. For example, the master controller may transmit a scan command to the slave controller to trigger each scan cycle such that left and right sensor data sets (e.g., temperature sensors) are generated by the left and right garments as they are worn, and the sensor data sets are substantially matched in time. Similarly, the garment-garment pairing may allow the left garment to synchronize its internal clock with the internal clock in the right garment, as described in further detail below. Furthermore, in some variations, among a paired set of garments (two or more garments paired to communicate with one another), not all of the garments are required to be additionally paired with one or more electronic devices (e.g., receiving station, mobile computing device) in order to communicate sensor data and other data collected by the paired set of garments. For example, in some variations, only a single garment in the paired set of garments may be paired with one or more electronic devices. In an exemplary variation including a left foot garment and a right foot garment paired with the left foot garment, only the left foot garment is paired with one or more electronic devices, and the controller in the left foot garment communicates sensor data generated by both the left foot garment and the right foot garment.

### Replacement Garments

In some variations, one or more replacement garments may be provided to a patient. Such replacement garments may be intended to replace existing garments, such as garments that are old or expired (e.g., beyond their intended lifetime of use), malfunctioning, depleted of power, etc. For example, a patient or other user may order a replacement garment (or set of replacement temperature-sensing garments) through a native application executing on a mobile computing device, or through a web portal rendered on another computing device that is linked to the profile of the user. Replacement garments may be paired with an electronic device in a similar manner as that described above. For example, replacement garments may be paired with a receiving station and/or a mobile computing device.

In some variations, a replacement garment may be paired with a receiving station and/or mobile computing device prior to shipment to a recipient (e.g., patient.). For example, prior to shipment of the replacement garment, an operator may associate a garment identifier and/or kit identifier (e.g., scan a kit identifier (e.g., QR code) arranged on a box to access UUID(s) of replacement garments within the box). The operator may upload the kit identifier and/or the garment identifier of the replacement garment to be associated with the patient profile (as denoted in order information for the replacement garment, for example), thereby pairing the replacement garment to one or more electronic devices accessing the patient profile, such as a receiving station or a mobile computing device. The replacement garment may then be provided to the patient or other user as already paired to the patient profile. Accordingly, in some variations, the operator may provide replacement garment(s) without requiring additional pairing steps by the patient, and without requiring additional replacement receiving stations already pre-paired to the replacement garments (i.e., paired prior to shipment).

### Inactive Mode and Determining Placement on Patient

After the one or more garments are paired to an electronic device (710), the garments may be worn by a patient for gathering sensor data. Generally, the method may include determining whether the patient is wearing the garment so as to transition the garment from an inactive mode to an active mode in which useful sensor data is gathered for subsequent analysis regarding a health condition of the patient. Determining when the garment is placed on the patient (whereupon the useful and meaningful sensor data may be collected) may, for example, be important for reducing error, such as by enabling the filtering or disregard of “junk” or “trash” sensor data (e.g., data that is unimportant or not representative of patient conditions).

In some variations, with reference to FIG. 6, a determination of whether the patient is wearing the garment (e.g., determining placement of the garment of the patient 630 and/or determining removal of the garment from the patient 650) may be performed without user input. For example, in a variation of monitoring a patient with a temperature-sensing garment, a determination of whether the patient is wearing the garment may be at least partially based on a combination of one or more triggers including temperature measurements and/or other sensor output such as motion, orientation, etc., as further described below. One advantage of automatically performing such determinations without user input is that a patient typically does not need to change his or her habit or routine with respect to putting on the garment in order to activate the garment's monitoring functionality. For example, if a patient is required to affirmatively provide input (e.g., pushing a button on the garment or on a displayed user interface) to confirm that the garment being worn and activate the garment's sensors, there is a risk of loss of data if the patient fails to provide the proper input (e.g., due to forgetfulness or user error), thereby contributing to ineffective monitoring of patient health. In contrast, because in some variations the methods described include automatic determination of garment placement, monitoring may commence without relying upon a change in patient behavior (e.g., patients can simply don the garment as part of a normal routine), which contributes to more consistent and reliable sensor collection, and better monitoring of patient health.

Triggers: Motion and/or Orientation

As shown in FIG. 7, the method 700 may include maintaining each garment in an inactive mode (712). Generally, a controller integrated into the garment may remain in an inactive mode in order to limit battery-usage when the temperature-sensing garment is not being worn. However, in some variations, the controller may initiate a transition to an active mode based at least in part on sensor data from the garment.

For example, in some variations, the garment includes one or more motion or orientation sensors (e.g., accelerometer, gyroscope, IMU, etc.). In one exemplary variation, the motion and/or orientation sensors are integrated into an anklet or housing of the garment. The motion and/or orientation sensors may couple to an interrupt pin on the controller of the garment, which may default to an inactive mode in which the controller executes a minimum of processes.

A potential transition of the garment to an active mode may be initiated at least in part on a detection of motion and/or a predetermined orientation of the garment (716). For example, motion and/or orientation signals from sensors in the garment may be periodically sampled (e.g., every ten minutes, every five minutes, every minute, every 30 seconds, every 10 seconds, etc.). As shown in FIG. 7, if detected motion (e.g., measured by an accelerometer) is not above a predetermined threshold and/or a detected orientation (e.g., measured by a gyroscope) does not satisfy a predetermined orientation condition corresponding to an expected orientation if the garment is worn (e.g., an ankle portion of a foot garment is upright or vertical), then the controller may interpret this as an indication that the garment is not in use (not placed on a patient). Accordingly, the garment may remain in an inactive mode (712).

If detected motion is above a predetermined threshold and/or detected orientation of the garment satisfies a predetermined orientation condition (e.g., an ankle portion of a foot garment is upright or vertical), then additional sensor information may be obtained and analyzed in order to corroborate an indication that the garment is in use. For example, in variations in which the garment is a temperature-sensing garment with at least one temperature sensor, if sufficient motion and/or predetermined orientation of the garment is detected, then a controller in the garment may scan the temperature sensor(s) in the garment (720) for further analysis as described below. Alternatively, in some variations, detection of sufficient motion and/or a predetermined orientation of the garment may be sufficient to indicate that the garment is in use.

### Trigger: Temperature Data

As shown in FIG. 7, the method 700 may include determine placement of the garment on a patient at least in part based on temperature measurements. Temperature measurements may be correlated to proximity to skin of the patient, due to the effect of body heat on the garment. Accordingly, temperature measurements by the sensors in the garment may be used at least in part to indicate whether the garment is being worn. For example, if signals from the temperature sensors indicate that the garment is not worn (as described below), then the controller may remain in the inactive mode (712) for at least a predetermined amount of time before repeating sensor scans (e.g., one minute). In contrast, if signals from the temperature sensors indicate that the garment is worn (as described below), then the controller may transition the garment to an active mode.

In some variations, the method may include scanning temperature sensor(s) in the garment (720), such as following a detection of sufficient motion and/or a predetermined orientation of the garment. However, it should be understood that the order of consideration of these triggers may be in any suitable order (e.g., temperature followed by motion and/or orientation). While in the inactive mode, the controller may intermittently or periodically scan the temperature sensors in the garment at a first sampling frequency (a nonzero frequency). For example, in the inactive mode, the controller may sample each temperature sensor (or at least one temperature sensor) at a first frequency such as between about once every minute and about once every 10 minutes, between about once every 3 minutes and about once every 7 minutes, or once about every 5 minutes. Furthermore, in some variations the first sampling frequency in the inactive mode may vary based on an environmental and/or prior use condition (e.g., increase sampling frequency if an internal clock timer indicates current time is a time when the patient is likely to be awake (e.g., between 6 AM and 11 PM, etc.) when the garment may be more likely to be placed on the patient, increase sampling frequency if the same garment has been determined to be placed on the patient at least once within the week, two weeks, or other predetermined time period, etc.). In an exemplary variation, one or more temperature sensors in the garment may output temperatures in the form of analog resistance values that the controller may convert into digital temperature values, or temperature data.

The controller may scan any number of temperature sensors in the garment at substantially the same time, in parallel. For example, the controller may scan at least a portion (e.g., all) of the temperature sensors in the garment at the same time. In another example, the controller may scan only one of the temperature sensors in the garment at one time, in series. Furthermore, the first sampling frequency may be the same for any number or portion of the temperature sensors in the garment, or may differ among at least two of the temperature sensors in the garment.

In some variations, while in the inactive mode, the controller may store temperature data in local memory and/or transmit temperature data to the user's mobile computing device via a wireless communication module, such as in real-time or asynchronously when the wireless communication module intermittently connects to the receiving station and/or the mobile computing device.

Generally, one or more of the temperature measurements obtained by scanning the temperature sensors in the garment (720) may be analyzed for an indicative temperature change in the garment (722). In some variations, the sensor data from each temperature sensor may be assessed individually and separately for an indicative temperature change over a sampling period. Alternatively, an average (e.g., mean, median, etc.) of the sensor data from multiple temperature sensors may be assessed for an indicative temperature change over a sampling period.

An indicative temperature change may take any suitable form. For example, an indicative temperature change in the garment may include a rising or upward trend (e.g., positive slope of rate of temperature change) in the individual or average temperature measurements of one or more sensors over a sampling scan period. As another example, an indicative temperature change in the garment may include a rate of temperature change (e.g., considering both sign and magnitude of the rate of temperature change) over a sampling scan period, and the controller may determine that the garment has been placed on the patient if the rate of temperature change satisfies a predetermined threshold rate of change. For example, the threshold rate of change may be between about 0.5 degree Celsius/second and about 5 degrees Celsius/second, or about 1 degree Celsius/second.

As another example, in some variations, an indicative temperature change in the garment may include a temperature gradient between discrete temperature sensors in the garment, and the controller may compare the temperature gradient to a threshold gradient pattern characteristic of the garment being worn (e.g., template temperature gradient patterns). As an illustrative example of determining garment placement based at least in part on temperature gradient, a one degree difference between a temperature sensor proximate a heel portion of a foot garment and a temperature sensor proximate a toe region of the foot garment may indicate that the patient is wearing the temperature-sensing garment.

As yet another example, in some variations, the controller may compare one or more of the absolute values of temperature measurements to a predetermined threshold (e.g., at least 35 degrees Celsius, or at least 37 degrees Celsius, near typical body temperature). If one or more of the absolute values of temperature measurements meets a predetermined threshold, the controller may then determine that the garment has been placed on the patient.

### Other Triggers

Other sensor data and/or other suitable triggers may, as an addition or alternative to any of motion, orientation, and temperature data (as described above), be relied upon by the controller to determine whether the garment is placed on a patient.

In some variations, the garment may additionally or alternatively include at least one magnet and a Hall effect sensor that are arranged relative to one another such that their relative positions change between when the garment is not placed on the patient and when the garment is placed on the patient. For example, in a variation in which the garment is a foot garment, at least one magnet may be arranged on a medial side of a leg region of the foot garment, and the Hall effect sensor may be arranged on a lateral side of the leg region (e.g., in the housing described above). In this example, when the garment is not placed on the patient (and the garment may, for example, be folded), the medial and lateral sides of the garment may be touching or otherwise in close proximity, which may be detectable by the Hall effect sensor due to its proximity and/or orientation to the at least one magnet. Conversely, when the garment is placed on the patient, the medial and lateral sides of the garment may be separated (e.g., due to placement of the patient's leg between the medial and lateral sides of the garment), which may also be detectable by the Hall effect sensor due to its separation from the at least one magnet. Accordingly, the Hall effect sensor's interactions with the magnet(s) may be used to help determine placement of the garment on the patient. It should be understood that other relative positions of the at least one magnet and the Hall effect sensor may be possible on the garment for this purpose (e.g., front and back surfaces of a garment, opposing sides of a foot region of a foot garment, etc.)

In some variations, the garment may additionally or alternatively include one or more pressure sensors placed at one or more contact locations of the garment (e.g., bottom or sole region of a foot garment). For example, if the garment is somewhat restrictive and elastic so as to conform to a body part when worn, the garment may be expected to experience at least a threshold amount of pressure if the garment is worn. Additionally or alternatively, forces applied to the garment during use (e.g., standing, sitting, sleeping, etc.) may be measured by one or more pressure sensors incorporated into suitable portions of the garment. Once in the active mode, the controller may regularly sample the pressure sensor(s) on such elastic or other suitable portions, such as just before each temperature scan cycle or once per another suitable interval (e.g., five or ten minute interval) to repeatedly confirm whether the garment is placed on the patient.

Additionally or alternatively, the garment may include one or more stretch sensors integrated into the garment. For example, one or more tension sensors or other stretch sensors may be added to the material (e.g., yarn) forming at least a portion of the garment (e.g., leg region of a foot garment) may detect when the garment is stretched or otherwise deformed to conform to a body part when worn. Similar to that described above, the controller may regularly sample the stretch sensor(s) to repeatedly confirm whether the garment is placed on the patient.

Additionally or alternatively, the garment may include at least one proximity sensor configured to detect the presence of skin or a body part inside the garment, which may indicate that the garment is currently being worn. For example, the proximity sensor may include a capacitive touch sensor facing the internal surface of the garment (e.g., integrated into a housing embedded into or installed over the exterior of the garment). As another example, the proximity sensor may include an IR sensor or other suitable proximity sensor. The proximity sensor may be coupled to an interrupt pin on the controller, which can default to a sleep state in which the controller executes a minimum of processes. Proximity to a massive object (e.g., a foot or a leg) may trigger a change in the output state of the proximity sensor from a binary LO voltage to a binary HI voltage (or vice versa), which can trigger the controller to transition from the sleep state to an active mode. Once in the active mode, the controller may regularly sample the proximity sensor, such as just before each temperature scan cycle or once per another suitable interval (e.g., ten-minute interval) to repeatedly confirm whether the garment is placed on the patient. Similarly, the controller may return to the inactive mode in response to a change in the output of the proximity sensor to the binary LO voltage, thus indicating that the garment is no longer worn, and/or in response to a deactivation command received from the patient's mobile computing device.

As yet another example, in some variations, the garment may additionally or alternatively include one or more moisture sensors configured to detect moisture from a patient when the garment is worn by the patient. For example, skin moisture sensors may be arranged on a suitable surface (e.g., on an internal surface) of the garment configured to detect moisture released from skin of the patient when the patient is wearing the garment. In some variations, additional reference moisture sensor(s) may be arranged on another surface of the garment of the garment that may provide a reference environmental measurement (e.g., an external surface of the garment configured to not be in contact with the patient when the patient is wearing the garment). For example, the reference moisture sensor(s) may be used to help distinguish between skin moisture sensor values that are elevated due to environmental humidity (a “false positive” reading of high skin moisture sensor values, where the skin moisture sensor values would be substantially similar to the reference moisture sensor values) and skin moisture sensor values that are elevated due to the garment being placed on the patient (a likely “true positive” reading of high skin moisture sensor values, where the skin moisture sensor values would be sufficiently different than the reference moisture sensor values).

In yet other variations, patient or other user input may indicate placement of the garment on the patient. For example, the controller may switch into the active mode in response to receipt of an activation input from the patient's mobile computing device (i.e., from the native application), or activating a mechanical input on the garment (e.g., button, switch, slide, etc.) to manually indicate to the controller that the garment is placed on the patient.

### Active Mode Measurements

In response to determining that the garment is placed on the patient, the controller may transition the garment to an active mode in which the controller scans sensors (e.g., temperature sensors) in the garment substantially continuously or intermittently at an increased, second scan frequency (740). The second sampling frequency may be greater than the first sampling frequency in the inactive mode, so as to provide substantially continuous monitoring via the one or more sensors in the garment. For example, the second sampling frequency may be at least five times, ten times, twenty times, or thirty times faster than the first sampling frequency. In some variations, in the active mode, the controller may sample each temperature sensor (or at least one temperature sensor) at a second sampling frequency such as between about once every second and about once every 5 minutes, between about once every minute and about once every 5 minutes, between about once every second and about once every minute, between about once every second and every 30 seconds, or about 10 seconds. In some variations, the second sampling frequency in the active mode may be substantially consistent over time. Alternatively, the controller may scan the set of temperature sensors at variable time intervals. For example, the controller may scan the set of temperature sensors once per hour while the controller determines that the user is at rest, such as indicated by outputs of an accelerometer or other motion sensor integrated into the garment or integrated into the mobile computing device. In another example, a patient may wear a left garment on a left foot and a right garment on a right foot, such that contralateral temperature measurements (e.g., at corresponding anatomical locations) may be obtained at substantially the same times. In this example, in response to a threshold temperature difference determined between two contralateral temperature (e.g., determined by a native application executed on a mobile computing device), a command may be transmitted to the left and right garments to sample the left and right temperature sensors at an increased rate. The left and right garments may, as an illustrative example, sample the sets of left and right temperature sensors at a rate of once per ten-minute interval (or other suitable interval) by default, but may sample the left and right temperature sensors at an increased rate of once per minute in response to receipt of such a command from the native application and/or in response to initiation of the active mode. Alternatively, only the left and right garment sensors responsible for the threshold contralateral temperature difference may be sampled at an increased sampling frequency.

In one variation, the controller may scan the set of sensors (e.g., temperature sensors) in response to an input entered by a user. For example, a patient or other user may manually trigger left and right garments to scan left and right temperature sensors, such as by selecting a “scan” option within the native application executing on the user's mobile computing device; the native application may then transmit scan commands to the left and right garments. Upon receipt of these scan commands, the left and right controllers may scan the left and right set of temperature sensors, respectively, and then transmit these temperature data back to the mobile computing device for analysis and/or presentation to the user at the native application, such as substantially in real-time.

The controller may also scan the set of sensors (e.g., temperature sensors) to collect a set of control or baseline sensor values over a period of time in which the state of the patient is known. For example, once a left garment, a right garment, and a mobile computing device are wirelessly paired and the native application confirms the battery charge state of the left and right garments, the native application may prompt the patient to assume a seated or prone position for a period of time (e.g., five minutes) while baseline or “control” temperature values are recorded. While outputs of an accelerometer or gyroscope integrated in the mobile computing device (or into the left garment or right garment) may help confirm that the patient is substantially motionless, the left and right garments may regularly sample the left and right temperature sensors, respectively (e.g., at a rate of about 1 Hz), and may upload the temperature data to the mobile computing device. Upon receipt, the native application may label the temperature data as baseline or control temperature values representing a baseline temperature patterns. For example, baseline data for a left foot may include a baseline temperature gradient of the patient's left foot in a nominal condition (e.g., known healthy condition). Such a temperature gradient may persist across the patient's left foot over time, even under changing environmental conditions such as walking on garments across a cool tile floor or walking in shoes across a hot asphalt road. Accordingly, as an illustrative example, the native application may compare temperature gradients across temperature values read from the left set of temperature sensors at a later date to the baseline temperature gradient represented by these control data to identify a change in the condition or health of the user's left foot, as described below. Additionally or alternatively, a baseline contralateral differential may be established from the baseline data.

In some variations, a controller may scan temperature sensors from multiple garments. For example, in some variations, a left controller in a left garment may scan temperature sensors integrated into a right garment (e.g., the second of a pair of garments) with temperature sensors arranged in a pattern mirroring that of the temperature sensors integrated into the left garment. In this example, the left controller may scan each of the temperature sensors integrated into both the left garment and the right garment at substantially the same time. In another example, the left controller may scan a particular pair of corresponding temperature sensors integrated into the temperature-sensing garments (e.g., a temperature sensor included in the left garment and a temperature sensor included in the right garment that occupy mirrored or corresponding positions within their respective patterns) at substantially the same time. By collecting temperature data from the left garment and the right garment at substantially the same times, the native application may discern, for example, between (i) changes in global temperatures of the user's feet due to motion or changing ambient conditions (or “noise”) and (ii) changes in local temperatures of regions of a foot which may indicate a medical condition of increasing risk to the user.

While in the active mode, the controller may store sensor data in local memory (742), such as for later transmission of sensor data to a paired mobile computing device via a wireless communication module, as described below. For example, the controller may store up to about seven weeks' worth of sensor data in local memory, or any suitable amount of data.

### Communicating Data

As shown in FIG. 7, the method may further include communicating sensor data to the one or more paired electronic devices (744). For example, in some variations, the method may include detecting a connection (e.g., wireless communication link) between the garment and the one or more electronic devices (743) such as a mobile computing device and/or receiving station. After detecting a connection (e.g., in response to detecting a connection), the controller may broadcast or otherwise communicate sensor data to the connected mobile computing device and/or receiving station. Furthermore, the controller may transmit other data (e.g., proximity, motion data) sampled from sensors integrated into the garment to the mobile computing device and/or receiving station, and may receive data (e.g., commands, scan cycle prompts, clock times, etc.) from the mobile computing device and/or receiving station. Subsequent to receiving sensor data, the paired electronic device (e.g., mobile computing device and/or receiving station) may transmit sensor data and/or other information to a remote computing system (760), such as a cloud computing system.

In some variations, a controller in the garment may manage communication of sensor data locally stored in the garment to a connected mobile computing device and/or receiving station. The communication of sensor data may be performed substantially in real-time (e.g., after each scan cycle of sampling sensors in the garment) or asynchronously with sensor measurement when the communication module connects to the mobile computing device. For example, sensor data may be communicated after each scan cycle of sampling sensors in the garment (e.g., broadcasting a scan cycle of sensor data including measurements taken by all sensors in a garment at a particular point in time), or intermittently (e.g., broadcasting a batch of scan cycles of sensor data including measurements taken by all sensors in a garment at multiple sequential points in time) such as after every fifth scan cycle or once per hour. In some variations, the local memory in the garment may store a buffer stream of sensor data so as to help avoid loss of sensor data in the event of a disconnection between the garment and the electronic device during a broadcast of sensor data.

In some variations, if a garment is paired with both a mobile computing device and a receiving station, the garment may preferentially broadcast sensor data directly to the native application as a primary receiver, and secondarily broadcast sensor data to a receiving station if the native application is not receptive (e.g., not open or running on the mobile computing device, the mobile computing device has low or depleted battery levels, etc.). For example, if a patient's mobile computing device (e.g., smartphone) has an active connection with the garment (e.g., the native application described herein is open and active in facilitating the connection) and is within range of the garment, the garment may broadcast sensor data directly to the mobile computing device. However, the connection between the garment and the mobile computing device may not always be active, such as if the patient accesses the native application on their mobile computing device infrequently or discontinuously. In such instances, the garment may instead broadcast sensor data to a receiving station paired to the garment when the garment is within range (e.g., five meters) of the receiving station, and the receiving station may subsequently upload the received sensor data to the patient profile. For example, the receiving station may be plugged into a wall and/or stationed within a user's residence and connected to a wireless and/or cellular network. When a garment is within range of the receiving station, the receiving station may receive a signal from the garment indicating the connection, and route this signal flag, alarm, or notification to a remote computer system and/or network. Additionally or alternatively, in some variations, if the garment is out of range of a receiving station, other active garments (e.g., a left garment) may cooperate to form a distributed network within the patient's vicinity (e.g., residence) and can collect and route the flag, alarm, or notification to a nearby wireless communication hub for distribution to the remote computer system.

Alternatively, the garment may preferentially broadcast to a receiving station as a primary receiver, and secondarily broadcast to a mobile computing device if a receiving station is not within range (or communication link is malfunctioning, etc.).

In some variations, the receiving station (and/or native application) may cross-reference garment identifiers (e.g., UUIDs) of garments broadcasting data with a registry of garment identifiers of garments that are paired to the receiving station and/or native application and recorded in the patient profile as described above. The receiving station and/or mobile computing device (or remote computing device analyzing received sensor data) may reject or ignore sensor data received from garments that are absent from the registry of paired garments, which may indicate that the garments broadcasting sensor data may instead be affiliated with another user, such as a spouse or another resident within the same household as the patient.

### Timestamp

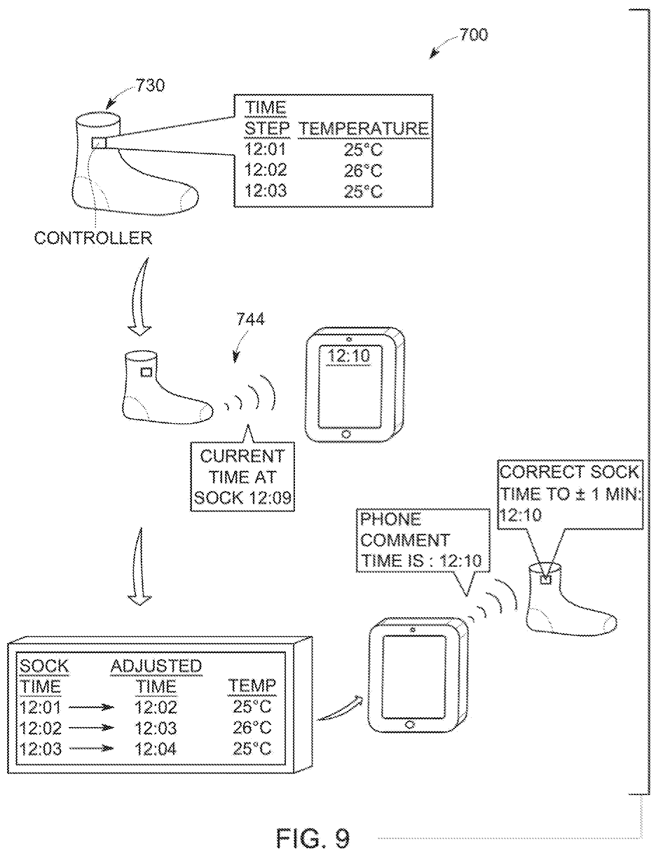

As shown in FIG. 9, the controller in the garment may maintain an internal timer that can operate substantially continuously throughout operation. The controller may record timestamps of each sensor measurement (e.g., temperature value) sampled by sensors integrated into the garment. In addition to broadcasting sensor data, the controller may broadcast these timestamps to a paired electronic device (e.g., a mobile computing device, receiving station, etc.).

In some variations, the controller may pair with the mobile computing device and/or receiving station, which may access a global (e.g., “atomic”) time over a wireless and/or a cellular network. In this example, the controller may calibrate the internal timer to align with the atomic time. However, the internal timer of the controller may drift (e.g., slow or quicken) over time and therefore, a current time recorded at the internal timer may differ from a current atomic time. To address such drift, when the controller pairs with the receiving station and/or the mobile computing device, the controller may query the receiving station or mobile computing device for a current atomic time. The controller may then account for any offset between an inaccurate timestamp previously indicated by the controller's internal timer and the corrected timestamp. For example, the controller may reset the internal timer to align with the current global time; determine a correctional offset in timestamps; retroactively correct timestamps of sensor data previously recorded based on the offset (e.g., add or subtract time according to the offset); and broadcast the temperature values and corrected timestamps to the receiving station and/or mobile computing device.

In another example, a garment may pair with a mobile computing device executing the native application as described herein, and the native application may synchronize a master clock maintained at the mobile computing device with discrete slave clocks integrated into the garment (or multiple garments, such as a left foot garment and a right foot garment). Once the master and slave clocks are synchronized in time, the controller in the garment may sample the garment's sensors such that all sensors are sampled at substantially the same time and share the same or similar timestamp. Furthermore, the controller in each of a pair of foot garments (such as a left foot garment and a right foot garment) may intermittently sample each garment's sensors such that pairs of sensor data sets generated by the left foot garment and the right foot garment may be substantially matched in time. Accordingly, in a variation involving a left temperature-sensing foot garment providing left foot temperature values and a right temperature-sensing foot garment providing right foot temperature values, the left and right foot garments may produce synchronized sets of left and right foot temperature values that may be analyzed as discussed below.

In another variation, one garment (e.g., a “left” garment) may wirelessly pair with a second garment (e.g., “right” garment), such as in an instance in which the mobile computing device is not available (e.g., is not within wireless range of the left and right garments). In this variation, the one garment may maintain an internal master timer and synchronize scan cycles with the other garment, and/or or trigger simultaneous scan cycles at the first and second garments as described above.

### Duplicate Garment Lockout

In some variations, when a controller in a first garment transitions into the active mode following detection of placement of the first garment on the patient, the controller may immediately ping the paired electronic device (e.g., receiving station or mobile computing device). Upon establishing an active communication link to the electronic device, the controller may upload any local data not previously transmitted to the electronic device and an indication that the first garment is currently in use. The controller may subsequently regularly broadcast sensor data for the first garment, such as at one-minute intervals. In this example, the receiving station (or mobile computing device or the remote computer system) may also lock out data collected by other duplicate garments of the same type from being stored in the user's profile while the first left garment is in the active mode. For example, if the first garment is a left foot garment configured to be worn on the left foot of the patient, data collected by other left foot garments, such as in a kit provided to the user, may be prevented from being stored in the electronic device, the patient's profile, etc. Locking out data from duplicate garment types may, for example, help ensure that sensor data analysis for monitoring the patient is limited only to data that was generated by garments being worn, thereby improving accuracy of the analysis and improving efficacy of patient monitoring.

When the controller later determines that the first garment is no longer being worn (e.g., when signals output by sensors in the first garment indicate that the garment is no longer moving and is cooling to below typical human body temperatures) and before the controller returns to the inactive mode, the controller may return a prompt to the electronic device indicating that the first garment is no longer in use. Once the first garment returns to the inactive mode, the lockout may be lifted and the receiving station (or the mobile computing device or remote computer system) may be open to collection and storage of sensor data provided by other garments of the same type as the first garment.

### Notifications and Other Features

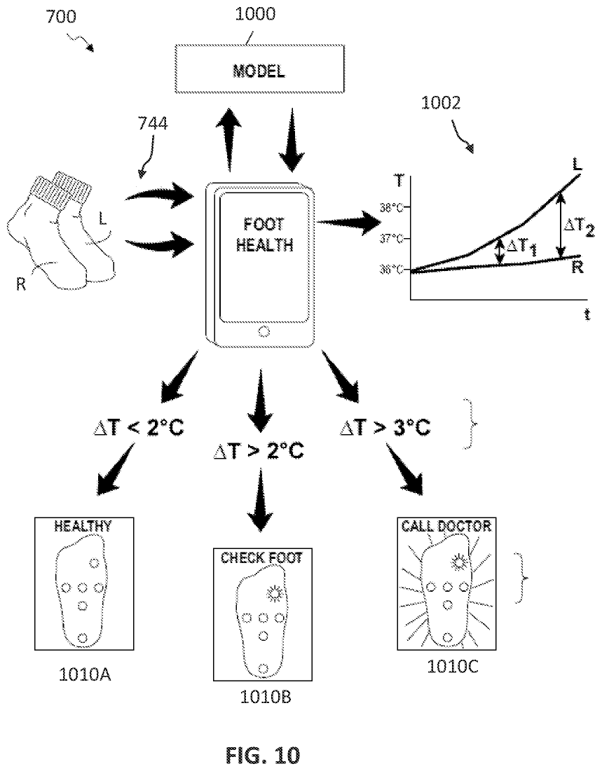

As shown in FIG. 10, a native application (e.g., executed and rendered on a mobile computing device) may present sensor data, diagnoses, garment function data, and/or other suitable information to the patient through a user interface executing within the native application.

For example, in some variations a monitoring system includes a left temperature-sensing garment configured to be worn on a left foot of a patient, and a right temperature-sensing garment configured to be worn on a right foot of a patient. When the left temperature-sensing garment is in the active mode, a controller in the left garment may sample temperature sensors in the left garment over time to generate a set of left foot temperatures in regions of the patient's left foot, and a controller in the right garment may sample temperature sensors in the right garment over time to generate a set of right foot temperatures in regions of the patient's right foot. The sets of left foot and right foot temperatures may be communicated (744) to a mobile computing device or other paired electronic device, and further uploaded to a remote computing system as described above.

The temperature data may be analyzed (e.g., by one or more processors in a paired mobile computing device, in a receiving station, and/or a remote computing system, etc.) to identify possible medical conditions present in one or both of the patient's feet, such as according to a model 1000. For example, in some variations, based on the model 1000, a native foot health application executing on the mobile computing device may compare and analyze these foot temperature data in real-time and/or over time to identify possible medical conditions present in one or both of the user's feet. In this example, the native foot health application may calculate a difference between left foot and right foot temperatures (1002) measured at like or corresponding regions on the patient's left and right feet at similar times (e.g., substantially simultaneously). The native application may further trigger a notification and/or identify a possible medical condition in the patient's feet if the calculated left-right foot temperature difference exceeds a threshold difference (e.g., if the temperature in a location on one foot exceeds the corresponding temperature in the other foot by at least a threshold difference). In some variations, different threshold differences may trigger different severities or classes of notifications. For example, the native application may trigger a first type of notification if the calculated temperature difference exceeds a first threshold difference, and may trigger a second type of notification indicating a more severe alert, if the calculated temperature difference exceeds a second threshold difference that is higher than the first threshold difference.

Furthermore, in some variations, a notification may be triggered based at least in part on duration or persistence of a calculated temperature difference exceeding a threshold difference (e.g., number of consecutive days that the excessive temperature difference between a left foot temperature and a right foot temperature persists). For example, a notification may be presented only if the excessive temperature difference persists at least two days, at least three days, or another suitable duration. Furthermore, different severities of notifications may be presented depending on persistence of the excessive temperature difference (e.g., persistence over two days may trigger a lower level notification such as a caution or warning, whereas persistence over three days may be trigger a higher level notification such as a direction for the patient to seek prompt medical attention).

The native application may present a notification to inform the patient, prompt one or more actions to respond to the possible medical condition (e.g., perform visual inspection or assessment of foot, take a picture and provide to a care provider, contact or visit a care provider, etc.). The notification may take any suitable form. For example, the notification may be a push notification on a mobile computing device administered via a native application, a text message, an email, a phone call, or the like.

Additionally or alternatively, in a variation relating to a system with a left temperature-sensing garment and a right temperature-sensing garment worn by a patient, the native application may display virtual representations of a left foot and/or a right foot overlaid with regions that may depict a form of notifications. For example, these regions may be color-coded according to the absolute or relative temperature values read from temperature sensors in corresponding positions within the left and right garments. For example, the native application may render a green circle (or other suitable shape) over each region of the virtual left foot or virtual right foot that corresponds to a temperature sensor location where temperature values are associated with a healthy condition (e.g., healthy condition representation 1010A), such as if temperature differences as measured between all left foot sensors and their corresponding right foot sensors is less than about 2 degrees Celsius. As another example, the native application may render a red circle (or other suitable shape) over each region of the virtual left foot or virtual right foot that corresponds to a temperature sensor location where temperature values are associated with an unhealthy condition. Additionally or alternatively, the native application may prompt a patient action such as a manual inspection of a foot (e.g., unhealthy condition representation 1010B indicating a location reflecting a temperature difference greater than a moderate threshold such as about 2 degrees Celsius), or contacting a care provider for medical attention (e.g., unhealthy condition representation 1010C indicating a location reflecting a temperature difference greater than a severe threshold such as about 3 degrees Celsius).

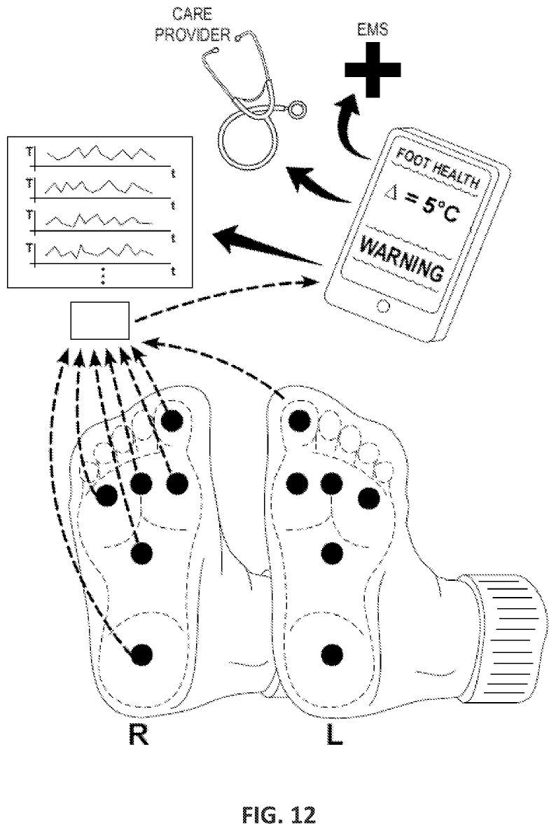

In some variations, as shown in FIG. 12, the native application may push temperature sensor data and/or medical condition predictions to a remote care provider, such as to a computing device or care provider portal associated with a nurse, doctor, or emergency responder. A care provider may thus view temperature data and/or medical condition predictions for the patient's feet in (near) real-time through the care provider portal and respond accordingly, such as by visiting the patient (e.g., for a care provider and patient occupying an assisted living facility, hospital, or clinic); calling the patient via telephone or video chat, scheduling an appointment with the patient at a doctor's office, dispatching an emergency responder to collect the patient from the patient's current location, or the like. Following the care provider's observation of the patient, the care provider portal may implement continue to monitor the patient through the monitoring systems and methods described herein. Furthermore, in some variations, the native app may write temperature sensor data and/or medical condition predictions directly to an electronic health record that is associated with the patient and stored in a local or remote database.

In some variations, the native application on a mobile computing device (or other electronic device) may enable tracking of patient symptoms. For example, the native application may present a text menu of selectable symptoms for a particular day, such as a burn, blister, bruising, a bug bite, a callous, loss of sensation, numbness, an open wound, redness, swelling, etc. Each symptom may be indicated for a particular body part (e.g., left foot, right foot) and location (e.g., top of foot, bottom of foot, side of foot). Similarly, as another example, the native application may allow a user to pictorially indicate symptoms (e.g., on a visual representation of a body part). Existence of such symptoms may help strengthen the prediction of a medical condition, and may be incorporated in a model for triggering notifications, such as by updating a medical condition model for the patient or by escalating a notification to a care provider to respond to a potential medical condition. Furthermore, such symptoms may be written to an electronic health record similar to that described above.

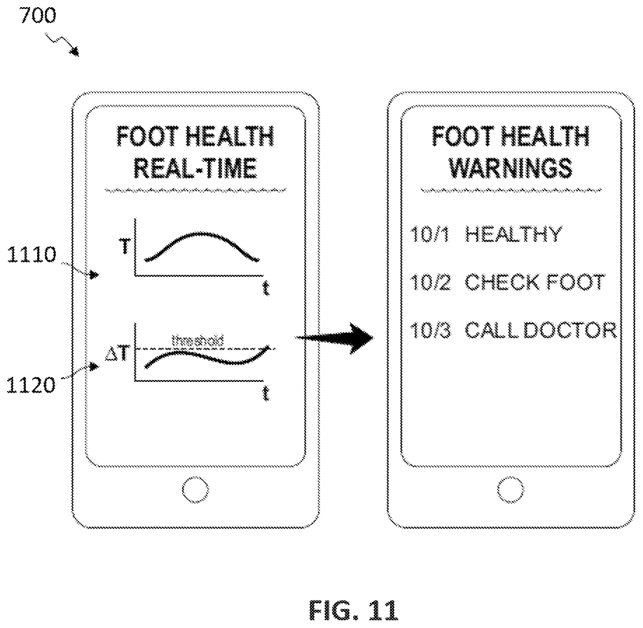

As shown in FIG. 11, the native application may additionally or alternatively render a virtual graph showing absolute temperatures 1110 or relative temperatures 1120 (e.g., differences between left foot temperatures and right foot temperatures) of each measured region of the patient's feet, or an average temperature of each of the patient's feet over time. For example, the native application may present sensor data to the patient through a virtual graph in order to visually communicate to the patient a trend in temperature of the patient's feet over hours, days, weeks, or months, which may indicate a change in the health of the patient's feet. For example, the native application may update the graph in real-time as data is received from sensors in the left and right garments.

However, the native application may serve temperature data, medical diagnoses, and/or notifications in any suitable way through the graphical user interface at the mobile computing device. Furthermore, as described above, the native application may upload sensor data to a remote computer system for remote processing and analysis, and the remote computer system may implement similar methods and techniques to process the data and can return medical diagnoses and/or prompts to the native application for presentation to the patient or to a web portal for presentation to a care provider, etc.

### Determining Removal of Garment

In some variations, as shown in FIG. 6, a method for monitoring may further include determining removal of the garment from the patient (660) (e.g., that the garment is no longer worn). The determination of garment removal may be based at least in part on conditions that are substantially opposite of the conditions described above for determining garment placement. For example, the controller of a garment may determine that a garment has been removed if motion and/or orientation sensors indicate a cease of motion of the garment and/or a garment orientation that correlates to storage of the garment (e.g., horizontal placement, such as if the garment is placed in a drawer). Additionally or alternatively, a determination of garment removal may be at least in part based on a detected temperature change indicative of garment removal (e.g., generally falling temperature values, a rate of negative change in temperature values exceeding a threshold rate of change, etc.). Furthermore, in some variations, a manual input from the patient on the garment and/or through a user interface (e.g., rendered by the native application on the mobile computing device) may be used to determine removal of the garment. Upon determining removal of the garment from the patient, the controller in the garment may return the garment to the inactive mode.

Furthermore, in variations including multiple garments (e.g., a left temperature-sensing foot garment, and a right temperature-sensing foot garment), a determination that one of the previously worn garments is removed may trigger an assumption that the other previously worn garments are also removed. For example, after a first garment is determined to be removed, a signal indicating this event may be sent (e.g., from a controller of the removed garment, just before the controller returns the garment to the inactive mode) to the paired electronic device (e.g., receiving station or mobile computing device). Upon receiving this signal, the paired electronic device may communicate to other paired garments still considered to be placed on the patient, and command their respective controllers to also return the garment to the inactive mode. In this manner, multiple garments may have a synchronized transition to the inactive mode, even though not all worn garments may be removed at the same time.

### Expiration of Garment

In another variation, the temperature-sensing garment may transmit the status of its power source to a paired receiving station and/or mobile computing device, which may track the charge state of the power source in the garment and prompt the patient to dispose of the temperature-sensing garment (e.g., such as through a graphical user interface rendered in the native application and/or haptic feedback such as a vibratory alert) when the power level drops below a threshold level. Furthermore, the controller of a garment may be configured to automatically disable temperature sensing and/or other functions of the garment upon expiration of the battery (e.g., after approximately six months).

Similarly, if a controller in a garment determines that a sensor in the garment is malfunctioning, the garment may upload all local sensor data to the receiving station or mobile computing device for storage in the patient's profile and then automatically disable itself from future use. For example, the garment may transmit to the receiving station a confirmation of malfunction and then disable itself from further temperature monitoring. The receiving station may receive this prompt from the garment and pass this information to the remote computer system, and the remote computer system may deactivate the garment in the patient's profile using a garment identifier (e.g., UUID) associated with the disabled garment. Furthermore, the system may automatically trigger shipment of a replacement garment to the patient using prepopulated patient information, and/or serve a notification to the patient that the garment has been disabled and that a replacement is on its way.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A system for monitoring a patient, comprising:
a garment comprising a controller and one or more temperature sensors; and
a receiving station configured to communicate with the garment over a wireless communication network,
wherein the garment is operable in:
an inactive mode wherein the controller samples the one or more temperature sensors to generate a first set of temperature measurements, and
an active mode, wherein the controller samples the one or more temperature sensors to generate a second set of temperature measurements and wherein the garment communicates the second set of temperature measurements to the receiving station,
wherein the controller transitions the garment from the inactive mode to the active mode based at least in part on the first set of temperature measurements.

2. The system of claim 1, wherein the controller automatically determines placement of the garment on the patient and transition the garment from the inactive mode to the active mode in response to determining placement of the garment on the patient.

3. The system of claim 2, wherein the garment further comprises an orientation sensor, and wherein the controller automatically determines placement of the garment on the patient based at least in part on a detected orientation of the garment satisfying a predetermined orientation condition.

4. The system of claim 2, wherein the garment further comprises a motion sensor, and wherein the controller automatically determines placement of the garment on the patient based at least in part on a detected motion of the garment satisfying a predetermined motion condition.

5. The system of claim 2, wherein the controller automatically determines placement of the garment on the patient based at least in part on a change within the first set of temperature measurements satisfying a predetermined temperature condition.

6. The system of claim 1, wherein when the garment is operated in the inactive mode, the controller samples the one or more temperature sensors at a first nonzero frequency, and when the garment is operated in the active mode, the controller samples the one or more temperature sensors at a second frequency greater than the first frequency.

7. The system of claim 1, wherein the garment is a first garment configured to be worn on a first foot of the patient and the system further comprises a second garment configured to be worn on a second foot of the patient.

8. The system of claim 7, wherein at least one of the first garment and the second garment comprises a plurality of temperature sensors arranged on a sole region of the garment.

9. The system of claim 1, wherein the garment is a sock.

10. The system of claim 1, wherein the receiving station further comprises a wireless communication module configured to communicate temperature measurements to a remote computing system over a second wireless communication network.

11. The system of claim 1, wherein the receiving station comprises a housing and a power supply.

12. The system of claim 11, wherein the housing comprises a visual status indicator.

13. A method for monitoring a patient, the method comprising:
pairing a garment to at least one electronic device, wherein the garment comprises one or more temperature sensors;
generating a first set of temperature measurements in an inactive mode using the one or more temperature sensors;
in response to a determination that the garment is placed on the patient, generating a second set of temperature measurements in an active mode using the one or more temperature sensors, and wirelessly communicating the second set of temperature measurements to the at least one electronic device.

14. The method of claim 13, wherein pairing the garment to the electronic device comprises pairing the garment to the electronic device using a garment identifier associated with the garment.

15. The method of claim 14, wherein pairing the garment to the electronic device comprises receiving a serial code or an image of a barcode label.

16. The method of claim 14, further comprising determining that the garment is placed on the patient.

17. The method of claim 16, wherein the determination that the garment is placed on the patient is based at least in part on detected motion of the garment, detected orientation of the garment, or both.

18. The method of claim 16, wherein the determination that the garment is placed on the patient is based at least in part on the first set of temperature measurements.

19. The method of claim 18, wherein determining that the garment is placed on the patient comprises determining that a change within the first set of temperature measurements satisfies a predetermined temperature condition.

20. The method of claim 13, wherein generating a first set of temperature measurements in the inactive mode comprises sampling the one or more temperature sensors at a first nonzero frequency, and wherein generating a second set of temperature measurements in the active mode comprises sampling the one or more temperature sensors at a second frequency greater than the first frequency.

21. The method of claim 13, wherein the at least one electronic device comprises a mobile computing device.

22. The method of claim 13, wherein the at least one electronic device comprises a receiving station.

23. The method of claim 13, further comprising wirelessly communicating the second set of temperature measurements from the electronic device to a remote computing system.

24. The method of claim 13, further comprising pairing a second garment to the at least one electronic device, wherein the second garment comprises one or more temperature sensors.

25. The method of claim 13, wherein the garment is a sock.

26. A method for automatically determining, without user input, whether a garment is placed on a user, the method comprising:
detecting an orientation of the garment using one or more orientation sensors in the garment;
receiving a set of garment temperature measurements from one or more temperature sensors in the garment; and
determining that the garment is placed on a user based at least in part on the detected orientation of the garment and the received garment temperature measurements.

27. The method of claim 26, wherein determining that the garment is placed on a user is based at least in part on the detected orientation of the garment satisfying a predetermined orientation condition.

28. The method of claim 26, further comprising determining a change in the garment temperature measurements over a sampling period, and determining that the garment is placed on a user is based at least in part on the determined change in the garment temperature measurements.

29. The method of claim 28, wherein the determination that the garment is placed on a user is based at least in part on a determined rise in garment temperature measurements over the sampling period.

30. The method of claim 26, further comprising detecting motion of the garment using one or more motion sensors in the garment, and wherein the determination that the garment is placed on a user is based at least in part on the detected motion of the garment.
